(19) United States
(12) Patent Application Publication
(10) Pub. No.: US 2023/0404778 A1
(43) Pub. Date: Dec. 21, 2023

(54) IMPLANTABLE SYSTEM COMPRISING A VARIABLE-VOLUME RESERVOIR

(71) Applicant: UROMEMS, GRENOBLE (FR)

(72) Inventor: Hamid LAMRAOUI, VAULNAVEYS LE HAUT (FR)

(21) Appl. No.: 18/035,531

(22) PCT Filed: Dec. 9, 2021
(86) PCT No.: PCT/FR2021/052249

§ 371 Date: May 5, 2023

(30) Foreign Application Priority Data
Dec. 10, 2020 (FR) FR2013004

(51) Int. Cl. A61F 2/48; A61F 2/00

(52) U.S. Cl. CPC A61F 2/484; A61F 2/004; A61F 2250/0003; A61F 2250/0013

(57) ABSTRACT

The invention describes a system that is implantable in a human or animal body, comprising: —a fluidic circuit comprising: —an inflatable element (3) containing a variable volume of a fluid; —a variable-volume fluid reservoir (5) comprising a fixed first part (10) and a movable second part (20); and—an actuator (8) mechanically coupled to the second part (20); wherein the second part (20) is also elastically deformable such that, when the second part (20) is fixed with respect to the first part (10), the second part (20) is designed to be mechanically deformed in response to a variation in pressure in the fluidic circuit, so as to alter the volume of the reservoir (5) in order to compensate for said variation in pressure in the fluidic circuit.

### FIELD OF THE INVENTION

The present invention relates to a system that is implantable in the human or animal body, comprising a fluid circuit with a variable-volume reservoir.

### PRIOR ART

Medical devices take the form of systems that are implantable in the body of a human or animal individual. In particular, such devices can correspond to artificial urinary sphincters used to combat urinary incontinence, gastric bands or rings suitable for restricting the stomach with a view to combating obesity, inflatable penile implants used for erectile prostheses, etc.

In a manner known per se, the implantable system can operate hydraulically and may comprise, in particular, a variable-volume fluid reservoir and an inflatable element containing a variable volume of fluid. The inflatable element is in fluidic connection with the variable-volume fluid reservoir, so as to be able to transfer the fluid from the reservoir to the inflatable element, and vice-versa.

In the case of an implantable occlusive system such as an artificial urinary sphincter, the inflatable element is an inflatable occlusive cuff capable of selectively closing an anatomical conduit such has a urethra in men, or a bladder neck in women. Fluid can be transferred from the reservoir to the cuff in order to increase the pressure exerted on the conduit, and conversely from the cuff to the reservoir in order to reduce the pressure exerted on the conduit. Hence, depending on the volume of fluid in the cuff, a greater or lesser pressure can be exerted on the anatomical conduit to be closed.

The injection and suction of fluid in the inflatable cuff necessary to close the anatomical conduit can be produced either manually and passively, such as for the artificial urinary sphincter with reference AMS800 marketed by American Medical Systems or the implant with reference ZSI375 marketed by Zéphyr, or automatically and actively (using an electrical power source, for example) for more developed implants.

WO 2016/083428 A1 describes such an implantable occlusive system comprising a variable-volume reservoir in fluidic connection with an occlusive cuff.

The fluid circuit of the implantable system is able to undergo a temporary and potentially sudden variation in pressure, in particular an overpressure of the inflatable element.

For example, in the case of artificial urinary sphincters, a physical activity or a change of posture of the individual in whom the system is implanted, for example when the individual is sat on a bicycle saddle, is able to cause an overpressure in the inflatable cuff. There is also a risk of overpressure during the introduction of a catheter into the urethra of the individual, since the needle of the catheter has a certain diameter and its introduction into the urethra causes an increase in the diameter of the urethra, and therefore an increase of the pressure in the cuff.

However, such pressure variations in the fluid circuit are to be avoided. In the case of artificial urinary sphincters, overpressures can damage the tissues around which the cuff is arranged, for example by causing lesions of these tissues. It is therefore necessary to rapidly control these overpressures, in order to limit the corresponding risk of tissue damage.

Pumping systems currently used in other fields suggest adding a deformable membrane combined with a spring, for example, in order to compensate overpressures in the system. However, these pumping systems would be difficult to include in an implantable system since they would add a great complexity to the structure and would pose problems of leak tightness, biocompatibility, compactness and efficiency. In addition, the deformation of the membrane could excessively modify the volume of the reservoir, which would cause a loss of precision in terms of the pressure imposed in the fluid circuit.

### DISCLOSURE OF THE INVENTION

An object of the invention is to provide an implantable system which can overcome the disadvantages of existing systems. In particular, the implantable system provided can rapidly, partially or entirely, compensate a variation in pressure in the fluid circuit.

Another object of the invention is to provide a sealed, biocompatible implantable system, having a minimum size and minimising the energy consumption necessary for compensating overpressure.

According to a first aspect, the invention relates to a system that is implantable in a human or animal body, comprising:
a fluid circuit comprising:
   an inflatable element containing a variable volume of a fluid;
   a variable-volume fluid reservoir, said reservoir comprising a stationary first part and a second part movable relative to the first part;
   a fluidic connection between the reservoir and the inflatable element; and
an actuator, mechanically coupled to the second part in order to selectively move the second part relative to the first part so as to vary a volume of the reservoir until a determined volume is attained;
   wherein the second part is also elastically deformable, such that, when the second part is stationary relative to the first part, the second part is adapted for being mechanically deformed at least in response to a variation in pressure in the fluid circuit, so as to adjust the volume of the reservoir relative to the determined volume in order to compensate, at least in part, said variation in pressure in the fluid circuit.

Some non-limiting features of the implantable system described above are the following, taken individually or in combination:
when the second part is stationary relative to the first part, the second part is in a rest configuration when the pressure in the fluid circuit is less than or equal to a limit pressure, the volume of the reservoir corresponding to the determined volume, and the second part is in a deformed configuration when the pressure in the fluid circuit is greater than the limit pressure, the deformation of the second part causing an increase in the volume of the reservoir adapted for re-establishing the limit pressure in the fluid circuit;
the implantable system further comprises a means for measuring pressure in the fluid circuit and a control unit, the control unit being adapted, when the pressure measured in the fluid circuit does not correspond to a setpoint pressure, for controlling an actuation of the actuator adapted for moving the second part so as to modify the volume of the reservoir in order to compensate the variation in pressure in the fluid circuit;
the inflatable element is an inflatable occlusive cuff;
the inflatable occlusive cuff is adapted for being arranged around an anatomical conduit in order to selectively close said anatomical conduit;
the second part comprises a movable wall and an elastically deformable bellows extending between the movable wall and the first part;
the actuator is adapted for controlling a linear movement of the movable wall of the second part, the bellows of the second part being adapted for extending or compressing according to said linear movement of the movable wall controlled by the actuator so as to vary a volume of the reservoir;
the first part forms an outer wall of the reservoir extending substantially coaxially with a longitudinal axis, the bellows of the second part forms an inner wall of the reservoir extending substantially coaxially with the longitudinal axis, and the actuator is adapted for moving the movable wall in translation along the longitudinal axis;
the bellows comprises a plurality of elastically deformable convolutions, and when the second part is stationary relative to the first part, the convolutions are adapted for being mechanically deformed so as to compensate the variation in pressure in the fluid circuit;
the convolutions are adapted for deforming in a direction of the longitudinal axis and/or in a radial direction with respect to the longitudinal axis;
the bellows comprises between 3 and 15 elastically deformable convolutions, preferably between 5 and 10 elastically deformable convolutions;
the bellows is made of titanium;
the bellows has a thickness between 0.01 mm and 0.3 mm, preferably between 0.03 mm and 0.15 mm;
the bellows has a stiffness constant between 0.2 and 15 N/mm, preferably between 1 and 4 N/mm;
the implantable system is configured to be implanted in a human or animal body in order to selectively close an anatomical conduit of said human or animal body, taken from at least one of the following conduits: a urethra, a gastric conduit, a colon or a rectum.

According to a second aspect, the invention relates to an assembly comprising an implantable system according to the first aspect and a remote control adapted for use by an individual in whom the system is implanted. The implantable system and the remote control comprise communication means adapted for communicating between them, and the remote control is adapted for controlling a movement of the second part by the actuator.

### DESCRIPTION OF THE FIGURES

Other features, goals and advantages of the present invention will become apparent on reading the following detailed description, given by way of non-limiting example, which will be illustrated by the following figures:

FIG. 1 shows a schematic view of an implantable system according to an embodiment of the invention.

FIG. 2 shows a schematic sectional view of an implantable system according to an embodiment of the invention.

FIGS. 3a and 3b show schematic sectional views of an implantable system according to an embodiment of the invention, respectively without and with deformation of the second part.

### DETAILED DESCRIPTION OF THE INVENTION

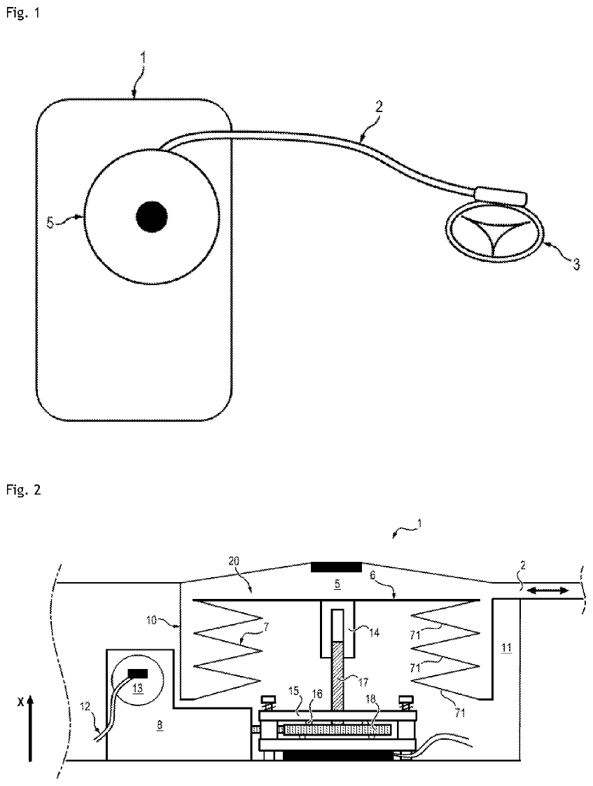

A system that can be implanted in a human or animal body is illustrated, by way of non-limiting example, in FIGS. 1, 2, 3a and 3b.

The implantable system comprises
a fluid circuit comprising:
   an inflatable element 3 containing a variable volume of a fluid;
   a variable-volume fluid reservoir 5, said reservoir 5 comprising a stationary first part 10 and a second part 20 that is movable relative to the first part 10;
   a fluidic connection 2 between the reservoir 5 and the inflatable element 3; and
an actuator 8 mechanically coupled to the second part 20 in order to selectively move the second part 20 with respect to the first part 10 so as to vary a volume of the reservoir 5 until a determined volume is attained.

The second part 20 is also elastically deformable, so that when the second part 20 is stationary relative to the first part 10, the second part 20 is adapted for being mechanically deformed at least in response to a variation in pressure in the fluid circuit, so as to adjust the volume of the reservoir 5 relative to the determined volume in order to compensate, at least in part, said variation in pressure in the fluid circuit.

The second part 20 of the fluid reservoir 5 of the implantable system is hence both movable and elastically deformable. The variation in pressure in the fluid circuit can be partially or entirely compensated.

The term “pressure in the fluid circuit” is used to designate the pressure established in each of the elements of the fluid circuit, in other words in the reservoir 5, in the inflatable element 3, and in the fluidic connection 2.

The term “setpoint pressure” is used to designate the desired pressure in the inflatable element 3, in other words in the fluid circuit.

The second part 20 is referred to as “stationary relative to the first part 10” in the absence of any actuating of the second part 20 by the actuator 8, in other words in a given position of the second part 20 relative to the first part 10.

A movement of the second part 20 by the actuator 8, the first part 10 being stationary, causes a variation in the volume of the reservoir 5. Hence, the second part 20 can be moved until a determined volume of reservoir 5 is attained, corresponding to the setpoint pressure.

The variable-volume reservoir 5 is adapted for being filled with a fluid. A variation in the volume of the reservoir 5 causes a variation in the pressure in the fluid circuit. More particularly, a reduction in the volume of the reservoir 5 leads to a transfer of fluid from the reservoir 5 to the inflatable element 3, and causes an increase in pressure in the fluid circuit. Conversely, an increase in the volume of the reservoir 5 leads to a transfer of fluid from the inflatable element 3 to the reservoir 5 and causes a reduction in pressure in the fluid circuit.

A deformation of the second part 20, in a given position of the second part 20, results in a variation in pressure in the reservoir 5, which can, in particular, be representative of a variation in pressure in the inflatable element 3. The second part 20 can, in particular, be adapted for deforming when the pressure in the reservoir 5 exceeds a limit pressure value greater than the setpoint pressure. The deformation of the second part 20 makes it possible to adjust the volume of the reservoir 5 relative to the determined volume, in order to compensate the variation in pressure in the fluid circuit, in particular to compensate an overpressure in the fluid circuit.

The deformation of the second part 20 is a mechanical deformation. Hence, the system removes the risk of measurement errors or breakdown of an electronic system, and does not assume any data processing by means of a particular software. The compensation of the variation in pressure is therefore carried out in a reliable and robust manner. In addition, the variation in pressure is mechanically and thus instantaneously compensated, without having to be detected beforehand by sensors, and without the action of any movement command of the second part 20. The implantable system therefore enables a faster compensation of any pressure variations in the fluid circuit, at the time when these pressure variations arise.

Hence, the implantable system is preserved and does not risk being damaged by a potential overpressure. The overpressure in the inflatable element 3 is transmitted to the reservoir 5, is then immediately absorbed by the deformation of the second part 20, so as to re-establish a pressure, in the fluid circuit, less than or equal to the limit pressure, preferably substantially corresponding to the setpoint pressure.

In particular, in the case where the implantable system is an artificial urinary sphincter, a temporary overpressure can arise in the inflatable element 3, for example during a physical activity or a change of posture of the individual in whom the system is implanted, or during introduction of a catheter in the urethra of the individual. The overpressure is compensated by the deformation of the second part 20, so that the conduit around which the inflatable element 3 is arranged, such as the urethra or the bladder neck, cannot be damaged by the application of a too large pressure by the inflatable element 3. The risk of lesions of the conduit due to the presence of the artificial urinary sphincter is thus limited.

The deformation of the second part 20 is elastic. Thus, once the variation in pressure has disappeared, the second part 20 resumes its initial shape, the volume of the reservoir 5 corresponding again to the determined volume.

### Inflatable Element

The inflatable element 3 can be made of biocompatible material, such as implantable silicone, implantable polyurethane, etc. The inflatable element 3 can be made from biocompatible elastomer, for example biocompatible silicone.

The inflatable element 3 can be an inflatable occlusive cuff, in particular when the implantable system is an artificial urinary sphincter. The inflatable occlusive cuff can be adapted for being arranged around an anatomical conduit in order to selectively close said anatomical conduit. The inflatable occlusive cuff 3 filled with fluid can be adapted for totally or partially surrounding the conduit to be closed.

Alternatively, the inflatable element 3 can be an inflatable penile implant, in particular when the implantable system is an erectile prosthesis.

### Fluidic Connection

The fluidic connection 2 may consist of a tube arranged between the reservoir 5 and the inflatable element 3. A first end of the tube opens into the reservoir 5 and a second end of the tube opens into the inflatable element 3.

The fluidic connection 2 can be made of biocompatible material, such as implantable silicone, implantable polyurethane, etc. The fluidic connection 2 can be made from biocompatible elastomer, for example biocompatible silicone.

### Variable Volume Reservoir

The implantable system is in a “static” mode when the second part 20 is stationary relative to the first part 10, i.e. in a given position of the second part 20. The implantable system is typically in the static mode when the volume of fluid in the reservoir 5 is adjusted to the desired volume of fluid in the inflatable element 3. In this static mode, the actuator 8 is inactive.

The implantable system is in a “dynamic” mode when the second part 20 is driven to move by the actuator 8 relative to the first part 10, typically when the volume of fluid in the reservoir 5 must be adjusted to obtain the desired volume of fluid in the inflatable element 3.

In the static mode, i.e. when the second part 20 is stationary relative to the first part 10, the second part 20 can be in a rest configuration when the pressure in the fluid circuit is a setpoint pressure less than or equal to a limit pressure, the volume of the reservoir 5 corresponding to the determined volume, and the second part 20 can be in a deformed configuration when the pressure in the fluid circuit is greater than the limit pressure, the deformation of the second part 20 causing an increase in the volume of the reservoir 5 adapted for re-establishing a pressure less than or equal to the limit pressure in the fluid circuit.

Hence, the second part 20 is in a rest configuration, i.e. does not undergo deformation, when the pressure in the fluid circuit is less than or equal to the limit pressure and the reservoir 5 has the determined volume. The deformation of the second part 20 being elastic, the second part 20 takes its undeformed shape when the volume of the reservoir 5 is equal to the determined volume and the pressure in the fluid circuit is less than or equal to the limit pressure.

The second part 20 can comprise a movable wall 6 and an elastically deformable bellows 7 extending between the movable wall 6 and the first part 10. The bellows 7 has a rigidity less than the rigidity of the movable wall 6. Hence, in the dynamic mode, the movement of the second part 20 results from the movement of the movable wall 6, and the deformation of the second part 20 results from the elastic deformation of the bellows 7.

The actuator 8 can be adapted for controlling a linear movement of the movable wall 6 of the second part 20, the bellows 7 of the second part 20 being adapted for extending or compressing according to said linear movement of the movable wall 6 controlled by the actuator 8 so as to vary a volume of the reservoir 5.

The second part 20 is therefore axially extendible or compressible, so as to vary the volume of the reservoir 5 in the dynamic mode. The movement of the second part 20 by the actuator 8 causes a corresponding linear movement of the movable wall 6 of the second part 20, and causes an extension or compression of the bellows 7 in the direction of linear movement, in particular in the direction of the longitudinal axis X. The movement of the movable wall 6, combined with the compression or extension of the bellows 7, causes a variation in the volume of the reservoir 5.

The first part 10 may comprise a cylindrical wall extending substantially coaxially with a longitudinal axis X, and a bottom wall adapted for closing the cylindrical wall at one of its ends. The cylindrical wall and the bottom wall, together define an outer wall of the reservoir 5.

The bellows 7 of the second part 20 can form an inner wall of the reservoir 5 extending substantially coaxially with the longitudinal axis X. The bellows 7 can be connected to the cylindrical wall of the first part 10 at a first end of the bellows 7, and be connected to the movable wall 6 at a second end of the bellows 7, opposite the first end. Hence, the cylindrical wall of the first part 10 closes the bellows 7 at its first end, and the movable wall 6 closes the bellows 7 at its second end.

The movable wall 6 of the second part 20 can extend perpendicularly to the longitudinal axis X. The actuator 8 is adapted for moving the movable wall 6 in translation along the longitudinal axis X.

The cylindrical wall of the first part 10 can substantially have the shape of a cylinder of revolution about the longitudinal axis X. The movable wall 6 can have a substantially disc shape, having a radius less than a radius of the cylindrical wall of the first part 10. The axis of revolution of the cylindrical wall of the first part 10 is preferably aligned with the longitudinal axis X and passes through the centre of the disc of the movable wall 6.

The actuator 8 can be chosen from any electromechanical system that can transform electrical energy into a mechanical movement with the output required to enable the movement with a force and at a required speed of the movable wall 6 of the variable volume reservoir 5. The actuator 8 can be, in particular, a piezoelectric actuator 8, an electromagnetic actuator 8 which may comprise an electromagnetic motor with or without brushes, coupled or not to a reduction gear, an electroactive polymer or a shape-memory alloy.

The movable wall 6 can be moved in translation along the longitudinal axis X, by the action of a drive screw 17 integral with the movable wall 6. The drive screw 17 can extend substantially along the longitudinal axis X. A position of the drive screw 17 may correspond substantially to a centre of the movable wall 6.

The actuator 8 is adapted for driving the rotation of the drive screw 17, for example by rotating a pinion. A rotation of the drive screw 17 around the longitudinal axis drives a movement of the movable wall 6 along the longitudinal axis X, the bellows 7 compressing or extending along the longitudinal axis X as a consequence.

The bellows 7 can comprise a plurality of elastically deformable convolutions 71. In the static mode, i.e. when the second part 20 is stationary relative to the first part 10, the convolutions 71 are adapted for being mechanically deformed so as to compensate the variation of pressure in the fluid circuit.

Hence, preferably, the deformation of the second part 20 corresponds to a deformation of the convolutions 71 of the bellows 7. In the event of variation in pressure in the variable-volume reservoir 5 in the static mode, in particular in the case of overpressure in the reservoir 5, the variation in pressure is transferred to the convolutions 71 of the bellows 7, which has the effect of changing the shape of the convolutions 71.

The bellows 7 can be in the form of accordion bellows 7. The convolutions 71 of the bellows 7 correspond to the folds of the accordion.

The convolutions 71 can be adapted for being arranged adjacent to one another in a direction of movement of the movable wall 6, in other words in the direction of the longitudinal axis X.

The convolutions 71 can be adapted for deforming in a direction of the longitudinal axis X and/or in a radial direction with respect to the longitudinal axis X.

Each convolution 71 can include a first portion 711 and a second portion 712 of convolution 71, connected to one another at a junction. The second portion 712 of a first convolution 71 can be connected to the first portion 711 of an adjacent second convolution 71, at a junction.

A space formed between the first portion 711 and the second portion 712 of the first convolution 71 defines a volume of the first convolution 71 which is outside of the reservoir 5. A space between the second portion 712 of the first convolution 71 and the first portion 711 of the adjacent second convolution 71 defines a volume which is part of the volume of the reservoir 5. A reduction in the volume of a convolution 71 causes a corresponding increase in the volume of the reservoir 5.

The first portion 711 and/or the second portion 712 of a convolution 71 can be in the shape of a substantially straight segment, or alternatively have a rounded or wavy shape, when the second part 20 is in the rest configuration. A rounded or wavy shape of the portions 711, 712 of a convolution 71 can facilitate the deformation of the convolution 71.

An angle can be defined between the first portion 711 and the second portion 712 of a convolution 71. In particular, the angle can correspond to an internal angle defined close to the junction between the first portion 711 and the second portion 712.

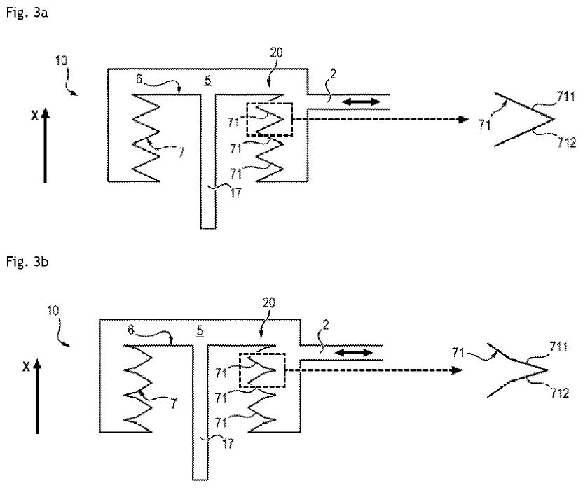

Each convolution 71 can substantially have the shape of a greater than sign “>” when the second part 20 is in a rest configuration, the two branches of the greater than sign “>” being formed respectively by the two portions 711, 712 of the convolution 71. FIG. 3a shows a non-limiting example in which the second part 20 is in the rest configuration and has such convolutions 71 in the shape of a greater than sign “>” formed by portions 711, 712 in the form of straight segments. The angle between the first portion 711 and the second portion 712 can substantially correspond to the angle formed between the two branches of the greater than sign “>” defined by the two portions 711, 712 of a convolution 71, close to a junction between the first portion 711 and the second portion 712.

When a variation in pressure arises in the reservoir 5, the overpressure affects the two portions 711, 712 of a convolution 71. Each portion 711, 712 of the convolution 71 is then able to deform.

In particular, in the event of overpressure in the fluid circuit, the convolutions 71 can “flatten”, the two portions 711, 712 of a given convolution 71 coming closer together, in other words the volume of the convolution 71 reducing. The angle formed between the two portions 711, 712 of the convolution 71 can be less than the angle formed between the two portions 711, 712 when the second part 20 is in a rest configuration.

Hence, in the event of such an overpressure in the reservoir 5, each convolution 71 is deformed so as to have a volume, defined by a space between its two portions 711, 712, less than the volume of the undeformed convolution 71. Consequently, the space extending between the second portion 712 of the first convolution 71 and the first portion 711 of the adjacent second convolution 71 is increased relative to the rest configuration. The volume of the reservoir 5 is therefore increased relative to the determined volume.

FIG. 3b shows a non-limiting example in which the second part 20 is in a deformed configuration due to an overpressure in the fluid circuit. The volume of the reservoir 5 is increased relative to the volume of the reservoir 5 when the second part 20 is in the rest configuration, as shown in FIG. 3a. The deformation of the convolutions 71 shown in FIG. 3b corresponds to a deformation substantially along the longitudinal axis X of the convolutions 71.

When the second part 20 is in the deformed configuration, in particular when an overpressure arises in the reservoir 5, the convolutions 71 can also deform in a radial direction, so as to again increase the volume of the reservoir 5 and hence compensate the overpressure.

The bellows 7 may be adapted for presenting a rigidity enabling a sufficient deformation to enable a compensation of any variation in pressure in the reservoir 5, and a rigidity sufficient that a movement of the second part 20 corresponds to a precise change in a volume of the variable-volume reservoir 5: hence, it is possible to impose, with precision, a quantity of fluid injected into the inflatable element 3 and a determined volume of the reservoir 5, while enabling compensation of a temporary variation in pressure in the reservoir 5. The lower the rigidity of the bellows 7, the more the bellows 7 deforms for a given pressure variation in the reservoir 5, and vice-versa.

The bellows 7 can have a stiffness constant between 0.2 and 15 newtons per millimetre (N/mm), preferably between 1 and 4 N/mm. The stiffness constant is a feature representative of the rigidity of the bellows 7. Such a stiffness constant between 0.2 and 15 N/mm, preferably between 1 and 4 N/mm, corresponds to a rigidity of the bellows 7 which conforms to the desired rigidity.

The rigidity of the bellows 7, represented where appropriate by the stiffness constant of the bellows 7, can be adjusted using one or other of a combination of features described below.

The bellows 7 may comprise between 3 and 15 elastically deformable convolutions 71, preferably between 5 and 10 elastically deformable convolutions 71. The greater the number of convolutions 71 of the bellows 7, the lower the rigidity of the bellows 7. A number of convolutions 71 between 3 and 15, preferably between 5 and 10, makes it possible to achieve a rigidity of the bellows 7 which conforms with the desired rigidity.

The bellows 7 can be made of titanium. Titanium enables a deformation adapted for the partial or total compensation of a temporary variation in pressure in the reservoir 5.

In addition, titanium is a biocompatible and airtight material. However, the reservoir 5 belongs to an implantable system. In the event of a leak of the fluid transmitted between the reservoir 5 and the inflatable element 3 or in the event of osmotic exchanges, for example in the fluidic connection 2 or the inflatable element 3, the fluid is able to pass into the body in which the system is implanted. Titanium makes it possible to prevent such leaks or osmotic exchanges from propagating from the inside of the housing 1 to the inside of the body and vice versa.

Alternatively, the bellows 7 can be produced from other materials, such as biocompatible stainless steel, or another material encapsulated in gold so as to be biocompatible.

The bellows 7 can be welded at the ends and at the base of the convolutions 71. Each portion 711, 712 of a convolution 71 can be welded at each of its ends to an adjacent portion 711, 712. Hence, the first portion 711 of a first convolution 71 can be welded at a first end to the second portion 712 of this same first convolution 71, and be welded at a second end, opposite the first end, to a second portion 712 of a second adjacent convolution 71. This feature improves the leak tightness of the bellows 7.

The bellows 7 can have a thickness between 0.01 mm and 0.3 mm, preferably between 0.03 mm and 0.15 mm. Such a thickness makes it possible to achieve a rigidity of the bellows 7 which conforms with the desired rigidity.

A person skilled in the art is able to adjust the geometry and dimensions of the bellows according to the limit pressure on the basis of which it must deform, while relying where appropriate on numerical simulations and/or experiments.

### Means for Measuring the Pressure in the Fluid Circuit

The implantable system may comprise a means for measuring the pressure in the fluid circuit, in other words a means adapted for measuring a pressure established in the reservoir 5, the inflatable element 3, and the fluidic connection 2.

The means for measuring the pressure in the fluid circuit may be a pressure sensor adapted for measuring a pressure in the reservoir 5. Alternatively, the means for measuring the pressure in the fluid circuit may be a force sensor adapted for measuring a force exerted on the movable wall 6 by the fluid contained in the reservoir 5. The surface area of the movable wall 6 being known, said force exerted on the movable wall 6 is representative of a pressure in the reservoir 5.

In some usage situations, the setpoint pressure defining the desired pressure in the inflatable element 3 can be chosen so as to correspond to a sufficient pressure in the inflatable element 3 to close the natural conduit without risking its damage, optionally taking into account the activity and/or posture of the patient. In other usage situations, the setpoint pressure can be chosen to be sufficiently low in order to release the natural conduit and allow urination.

In any event, the setpoint pressure is less than the limit pressure above which the second part 20 deforms. The value of said limit pressure can be dependent on the position of the movable wall 6 and/or the setpoint pressure.

### Control Unit

The implantable system can comprise a control unit.

The control unit can be configured to determine a volume of reservoir 5 corresponding to the setpoint pressure, and to control the actuator 8 so as to move the second part 20 of the reservoir 5 until attaining the determined volume of the reservoir 5 corresponding to the setpoint pressure.

More particularly, in the example illustrated in FIG. 2, the control unit is adapted for sending an order to operate the motor of the electromagnetic actuator 8 in one direction or the other, according to whether an increase or a reduction in volume of the reservoir 5 is required, the implantable system then being in the dynamic mode.

The implantable system can switch from the dynamic mode to the static mode, the second part 20 being in a given position, for example when the inflatable element 3 is inflated or when the determined volume of the reservoir 5 is attained.

Alternatively or in addition, a movement of the second part 20 can be controlled, for example, by the individual in whom the system is implanted. For example, in the case of an artificial urinary sphincter, when the individual wishes to urinate, he can control a movement of the second part 20 so as to increase the volume of the reservoir 5, which has the effect of causing a transfer of the fluid from the occlusive cuff 3 into the reservoir 5, thus reducing the pressure exerted by the cuff 3 on the conduit to be closed.

The control unit can, alternatively or in addition, be adapted for controlling a movement of the second part 20, on the basis of a pressure measured by the pressure measuring means. In particular, the control unit can be adapted, when the pressure measured in the fluid circuit does not correspond to a setpoint pressure, for controlling an actuation of the actuator 8 adapted for moving the second part 20 so as to modify the volume of the reservoir 5 in order to compensate the variation in pressure in the fluid circuit.

Hence, a variation in pressure in the fluid circuit, such as an overpressure, is first compensated by the deformation of the second part 20, this compensation being mechanical, immediate and direct. The compensation can be partial or total.

The compensation through the deformation of the second part 20 is performed during a period adapted for allowing the pressure measurement means to detect the variation in pressure in the fluid circuit and to allow the control unit to adjust a movement command of the second part 20 as a consequence. Hence, the variation in pressure in the reservoir 5 is reduced, or even the pressure in the reservoir 5 remains substantially constant, during the detection and adjustment of the command, due to the deformation of the second part 20.

Secondly, the variation in pressure in the fluid circuit is compensated by the movement of the second part 20 relative to the first part 10, the implantable system switching into dynamic mode. Said movement of the second part 20 is controlled by the control unit by means of the actuator 8. The second part 20 can be moved until the pressure in the fluid circuit corresponds to the setpoint pressure.

Then, the movement of the second part 20 by the actuator 8 is stopped, the implantable system switching into static mode.

In particular, when the pressure measured in the fluid circuit is greater than the limit pressure, in other words in the event of overpressure in the fluid circuit, the control unit can be adjusted to control, by means of the actuator 8, a movement of the second part 20 adapted for increasing the determined volume of the reservoir 5, in view of re-establishing, in the fluid circuit, a pressure corresponding to the setpoint pressure, which is less than or equal to the limit pressure.

### Casing

The variable-volume reservoir 5 and the actuator 8 can be incorporated in a sealed biocompatible casing 1 intended to be implanted in the body of an individual. The control unit and/or the pressure measuring means in the fluid circuit can also be incorporated in the casing 1.

The variable-volume reservoir 5 can, more particularly, be defined by the bellows 7 facing the first part 10, the movable wall 6 and the first part 10. The reservoir 5 further comprises an orifice for transferring fluid from and to the exterior of the reservoir 5 to the cuff 3 via the fluidic connection 2.

The casing 1, in particular the internal volume 11 of the casing 1 surrounding the reservoir 5, contains a gas, for example an inert gas.

The casing 1 can be made of titanium. Titanium being an airtight and biocompatible material, it can be used to protect elements arranged in the casing 1, in particular electronic components such as the control unit, potential sensors, etc., from the external environment.

The casing 1 is sealed to avoid any transfer of fluid or gas from or to the intracorporeal environment.

The casing 1 is made of a biocompatible material and can, for example, be made of implantable titanium and sealed by laser welding. A leak check can be carried out, in particular using helium, to ensure the total leak tightness of the casing 1 during the period for which the device is implanted.

FIG. 2 shows an example of an implantable system comprising a variable-volume reservoir 5 and an electromagnetic actuator 8 incorporated in a casing 1.

The actuator 8 comprises a motor 13 coupled to a reduction gear. A connector 12 makes it possible to supply power to the motor 13 according to an operating order of the motor.

The reduction gear is coupled to a toothed wheel 18 which is itself coupled to the drive screw 17, so as to transmit the torque and rotation of the shaft of the motor 13 to the drive screw 17.

The drive system further comprises a nut 14 coupled to the drive screw 17 and rotatable on a double-acting thrust ball bearing around the axis of the screw 17 under the effect of an action driven by the actuator 8. The movable wall 6 is coupled to the drive screw 17 via a nut 14, the nut 14 being integral with the movable wall 6 and having an internal thread cooperating with the thread of the screw 17.

Hence, a rotation of the nut 14 drives the screw 17 in translation only, in the direction of movement of the movable wall 6, which has the effect of moving the movable wall 6 in translation in a direction parallel to the axis of the screw 17, in other words in the direction of the longitudinal axis X. The direction of movement of the movable wall 6 depends on the direction of rotation of the motor 13.

The toothed wheel 18 is housed in a block 15 by means of ball bearings 16 which allow its rotation in the block 15.

### Assembly

The implantable system described above, an example of which is shown in FIG. 1, can be configured to be implanted in a human or animal body in order to selectively close an anatomical conduit of said human or animal body, taken from at least one of the following conduits: a urethra, a gastric conduit, a colon or a rectum.

An assembly may comprise an implantable system such as described above, and a remote control adapted to be used by an individual, for example by an individual in whom the system is implanted.

The implantable system and the remote control comprise communication means adapted for communicating between them. The remote control is adapted for controlling a movement of the second part 20 by the actuator 8, in particular on the basis of a command transmitted by the communication means of the remote control to the communication means of the implantable system.

The communication means of the implantable system can be incorporated in the casing 1.

Other embodiments are possible and a person skilled in the art can easily modify the modes or embodiments disclosed above or envisage others while remaining within the scope of the invention.

1. A system that is implantable in a human or animal body, comprising:
a fluid circuit comprising:
an inflatable element containing a variable volume of a fluid;
a variable-volume fluid reservoir, said reservoir comprising a stationary first part and a second part movable relative to the first part;
a fluidic connection between the reservoir and the inflatable element; and
an actuator, mechanically coupled to the second part in order to selectively move the second part relative to the first part so as to vary a volume of the reservoir until a determined volume is attained;
wherein the second part is also elastically deformable, such that, when the second part is stationary relative to the first part, the second part is adapted for being mechanically deformed at least in response to a variation in pressure in the fluid circuit, so as to adjust the volume of the reservoir relative to the determined volume in order to compensate, at least in part, said variation in pressure in the fluid circuit.

2. The implantable system according to claim 1, wherein, when the second part is stationary relative to the first part, the second part is in a rest configuration when the pressure in the fluid circuit is less than or equal to a limit pressure, the volume of the reservoir corresponding to the determined volume, and the second part is in a deformed configuration when the pressure in the fluid circuit is greater than the limit pressure, the deformation of the second part causing an increase in the volume of the reservoir adapted for re-establishing the limit pressure in the fluid circuit.

3. The implantable system according to claim 1, further comprising a means for measuring pressure in the fluid circuit and a control unit, the control unit being adapted, when the pressure measured in the fluid circuit does not correspond to a setpoint pressure, to control an actuation of the actuator adapted for moving the second part so as to modify the volume of the reservoir in order to compensate the variation in pressure in the fluid circuit.

4. The implantable system according to claim 1, wherein the inflatable element is an inflatable occlusive cuff.

5. The implantable system according to claim 4, wherein the inflatable occlusive cuff is adapted for being arranged around an anatomical conduit in order to selectively close said anatomical conduit.

6. The implantable system according to claim 1, wherein the second part comprises a movable wall and an elastically deformable bellows extending between the movable wall and the first part.

7. The implantable system according to claim 6, wherein the actuator is adapted for controlling a linear movement of the movable wall of the second part, the bellows of the second part being adapted for extending or compressing according to said linear movement of the movable wall controlled by the actuator so as to vary a volume of the reservoir.

8. The implantable system according to claim 6, wherein the first part forms an outer wall of the reservoir extending substantially coaxially with a longitudinal axis, wherein the bellows of the second part forms an inner wall of the reservoir extending substantially coaxially with the longitudinal axis, and wherein the actuator is adapted for moving the movable wall in translation along the longitudinal axis.

9. The implantable system according to claim 6, wherein the bellows comprises a plurality of elastically deformable convolutions, and wherein, when the second part is stationary relative the first part, the convolutions are adapted for mechanically deforming so as to compensate the variation in pressure in the fluid circuit.

10. The implantable system according to claim 8, wherein the bellows comprises a plurality of elastically deformable convolutions, wherein, when the second part is stationary relative the first part, the convolutions are adapted for mechanically deforming so as to compensate the variation in pressure in the fluid circuit and wherein the convolutions are adapted for deforming in a direction of the longitudinal axis and/or in a radial direction with respect to the longitudinal axis.

11. The implantable system according to claim 9, wherein the bellows comprises between 3 and 15 elastically deformable convolutions, preferably between 5 and 10 elastically deformable convolutions.

12. The implantable system according to claim 6, wherein the bellows is made of titanium.

13. The implantable system according to claim 6, wherein the bellows has a thickness between 0.01 mm and 0.3 mm, preferably between 0.03 mm and 0.15 mm.

14. The implantable system according to claim 6, wherein the bellows has a stiffness constant between 0.2 and 15 N/mm, preferably between 1 and 4 N/mm.

15. The implantable system according to claim 1, configured to be implanted in a human or animal body in order to selectively close an anatomical conduit of said human or animal body, taken from at least one of the following conduits: a urethra, a gastric conduit, a colon or a rectum.

16. An assembly comprising an implantable system according to claim 1 and a remote control adapted for being used by an individual in whom the system is implanted, wherein the implantable system and the remote control comprise communication means adapted for communicating between them, wherein the remote-control is adapted for controlling a movement of the second part by the actuator.
